(19) United States
(12) Patent Application Publication
(10) Pub. No.: US 2015/0133333 A1
(43) Pub. Date: May 14, 2015

(54) COMPOSITIONS AND METHODS FOR DETECTING COMPLICATED SARCOIDOSIS

(71) Applicant: The Board of Trustees of the University of Illinois, Urbana, IL (US)

(72) Inventors: Joe G.N. Garcia, Tucson, AZ (US); Tong Zhou, Tucson, AZ (US); Wei Zhang, Chicago, IL (US); Roberto Machado, Hinsdale, IL (US)

(21) Appl. No.: 14/483,803

(22) Filed: Sep. 11, 2014

Related U.S. Application Data
(60) Provisional application No. 61/877,129, filed on Sep. 12, 2013.

(51) Int. Cl. C12Q 1/68

(52) U.S. Cl. CPC C12Q 1/6883; C12Q 2600/156; C12Q 2600/158
USPC 506/9; 506/16; 536/24.31; 435/6.11

(57) ABSTRACT

Disclosed are kits and methods for diagnosing a person with, or assessing a person's risk for developing, sarcoidosis and/or complicated sarcoidosis.

### CROSS-REFERENCE TO RELATED APPLICATIONS

This application claims the benefit of U.S. Provisional Application No. 61/877,129, filed Sep. 12, 2013, which is incorporated by reference in its entirety.

### STATEMENT REGARDING FEDERALLY SPONSORED RESEARCH OR DEVELOPMENT

This invention was made with government support under NIH grants NHLBI HL58094, R01HL112051, HL68019, HL83870, U01 HL105371-01, RC2 HL101740-01, and NHLBI K23HL098454. The United States government has certain rights in this invention.

### INTRODUCTION

Sarcoidosis is a systemic inflammatory and non-caseating granulomatous disease of unknown origin that can damage multiple organs, including lungs, lymph nodes, skin, eyes, liver, heart, and brain.

Although sarcoidosis may spontaneously appear or disappear, approximately 20% of affected individuals experience progressive disease with respiratory, cardiac, or nervous system involvement. Complicated sarcoidosis is defined as exhibiting either cardiac manifestations (e.g., ventricular arrhythmias), neurologic involvement (e.g., with evidence of hyperdense MRI lesions), or deteriorating lung function (e.g., forced vital capacity (FVC)<50%).

The development and prognosis of sarcoidosis varies among certain racial and gender populations. The population group with the highest incidence rates of sarcoidosis is African American women. Genetic and non-genetic factors (e.g., age, exposure to certain environmental stimuli) affect disease risk. Genetic variation significantly contributes to sarcoidosis with cases five times more likely than control subjects to report an affected sibling or parent.

The assessment of sarcoidosis susceptibility in specific high-risk populations and the identification of sarcoidosis patients at risk for complicated, progressive disease remains a challenge.

There is a need in the art for sarcoidosis biomarkers to identify individuals with complicated sarcoidosis and to identify patients at risk for increased morbidity and mortality as a consequence of complicated sarcoidosis. The present invention addresses that need.

### SUMMARY OF THE INVENTION

One object of certain embodiments of the present invention is to provide kits for diagnosing a person with, or assessing the individual's risk for developing, sarcoidosis.

Another object of certain embodiments of the present invention is to provide methods for diagnosing a person with, or assessing the individual's risk for developing, sarcoidosis.

In certain embodiments, the kits and methods may be used to determine whether a person has sarcoidosis or complicated sarcoidosis.

In certain embodiments, the kit consists essentially of probes for measuring expression levels of one or more genes listed in Table 3. In certain embodiments, the kit consists essentially of probes for detected the presence or absence of one or more single nucleotide polymorphisms listed in Table 5.

In certain embodiments, the methods involve measuring the expression levels of one or more genes listed in Table 3 using a kit of the invention.

In certain embodiments, the methods involve detecting the presence or absence of one or more single nucleotide polymorphisms listed in Table 5 using a kit of the invention.

The present invention and its attributes and advantages will be further understood and appreciated with reference to the detailed description below of presently contemplated embodiments, taken in conjunction with the accompanying drawings.

### BRIEF DESCRIPTION OF THE DRAWINGS

Generally, FIG. 1A-FIG. 1D illustrate the certain elements of identifying gene signatures in sarcoidosis.

FIG. 1A illustrates enriched pathways among complicated sarcoidosis-associated genes. The top ranking KEGG pathways are listed for each population. The red line indicates the cutoff of significance (adjusted p-value<0.05). The number of genes in each pathway is shown beside the pathway name. The “AA” group illustrated in FIG. 1A(i) is “African Americans” and the “EA” group illustrated in FIG. 1A(ii) is “European descent ancestry”.

FIG. 1B illustrates a heatmap of patients with complicated sarcoidosis and healthy controls. Red represents increased gene expression; Blue represents down-regulation. “++”: patients with complicated sarcoidosis; “−”: healthy controls.

FIG. 1C illustrates principal component analysis on expression values of the 20-gene signature. The principal component 1 with eigenvalue is plotted on the X-axis, while principal component 2 with eigenvalue is plotted on the Y-axis. The graph in FIG. 1C(i) illustrates information regarding patients with complicated sarcoidosis and healthy controls. The graph in FIG. 1C(ii) illustrates information regarding patients with complicated sarcoidosis, uncomplicated sarcoidosis and healthy controls. The graph in FIG. 1C(iii) illustrates information regarding patients with complicated sarcoidosis and uncomplicated sarcoidosis. The term “HC” stands for “healthy controls”. The term “US” stands for “patients with uncomplicated sarcoidosis”; and the term “CS” stands for patients with complicated sarcoidosis.

FIG. 1D illustrates a comparison between the 20-gene signature and the TCR/JS/CCR signaling pathway gene signature. The distribution of prediction accuracy is based on 1,000 times of five-fold cross-validation. The dashed lines indicate the average classification accuracy for the 20-gene signature or the TCR/JS/CCR signaling pathway gene signature. FIG. 1D(i) illustrates information regarding all sarcoidosis patients versus healthy controls. FIG. 1D(ii) illustrates information regarding patients with complicated sarcoidosis versus patients with uncomplicated sarcoidosis.

FIG. 2A through FIG. 2T illustrate a box plot of expression of the principal component analysis on expression values of the 20 signature genes. The phrase “HC: means healthy controls, the phrase “US” means patients with uncomplicated sarcoidosis; and the phrase “CS” means patients with complicated sarcoidosis. The log₂-transformed expression values is plotted on the Y-axis.

### DETAILED DESCRIPTION

Certain diseases and conditions, such as sarcoidosis, are more likely to occur in people having certain genetic polymorphisms or having altered gene expression, i.e., increased or decreased expression of certain genes, relative to people without the disease or condition. Accordingly, identifying such genetic polymorphisms or altered gene expression is valuable in identifying patients at risk of developing sarcoidosis. Also, since sarcoidosis is not easily diagnosable, identifying genes associated with sarcoidosis could also assist with diagnosis.

Genes are made up of deoxyribonucleic acid (DNA), genetic material which, when expressed, produces a gene product, such as a messenger ribonucleic (mRNA), which in turn may be translated to produce a protein. Whether and when a certain gene is expressed may be controlled by other genes. Levels of mRNA expressed may be controlled by expression quantitative trait loci (eQTLs).

Defining eQTLs in the form of single nucleotide polymorphisms (SNPs) and profiling gene expression in peripheral blood mononuclear cells (PBMCs) allows identification of gene expression signatures as genomic biomarkers associated with sarcoidosis, thereby advancing personalized risk assessment for developing complicated sarcoidosis.

The present study was designed to identify novel genomic biomarkers by comparing genome-wide gene expression data in African American (AA) and European descent ancestry (EA) sarcoidosis cases. A universal gene signature that differentiates sarcoidosis patients from healthy controls and distinguishes complicated sarcoidosis (pulmonary (FVC<50%), cardiac, or neurologic sarcoidosis) from uncomplicated sarcoidosis was identified as described below. This gene signature was found to have superior in prediction accuracy in each of the AA and EA populations when compared to a second signature comprised of genes within the T cell receptor-innate immunity pathway, which includes genes previously found to be associated with sarcoidosis. These signatures distinguished sarcoidosis patients from idiopathic pulmonary fibrosis (IPF) cases with signature validation provided by significant association of genetic variants within signature genes with sarcoidosis susceptibility. These results highlight the utility of peripheral blood molecular gene signatures as valuable biomarkers for predicting individuals at risk for complicated sarcoidosis and for facilitating individualized therapies in this enigmatic disorder.

As described in the examples below, genome-wide peripheral blood gene expression analysis was used to identify a 20-gene sarcoidosis biomarker signature distinguishing sarcoidosis (n=39) from healthy controls (n=35, 86% classification accuracy) and which served as a molecular signature for complicated sarcoidosis (n=17). As aberrancies in T cell receptor (TCR) signaling, JAK-STAT (JS) signaling, and cytokine-cytokine receptor (CCR) signaling are implicated in sarcoidosis pathogenesis, a 31-gene signature comprised of T-cell signaling pathway genes associated with sarcoidosis (TCR/JS/CCR) was compared to the unbiased 20-gene biomarker signature but proved inferior in prediction accuracy in distinguishing complicated from uncomplicated sarcoidosis. Additional validation strategies included significant association of SNPs in signature genes with sarcoidosis susceptibility and severity (unbiased signature genes—CX3CR1, FKBP1 A, NOG, RBM12B, SENS3, TSHZ2; T cell/JAK-STAT pathway genes such as AKT3, CBLB, DLG1, IFNG, IL2RA, IL7R, ITK, JUN, MALT1, NFATC2, PLCG1, SPREDI.

The present invention includes novel compositions and methods for determining whether a person has, or is at risk for developing, sarcoidosis and/or compositions and methods for predicting prognosis, e.g., mortality or risk of developing complicated sarcoidosis, of a person individual with sarcoidosis. Further, the identification of genetic loci and SNPs associated with sarcoidosis contributes to the understanding of sarcoidosis pathogenesis and provides potential targets for novel treatments.

The compositions and methods of the present invention may be used determining whether a person has or is at risk of developing sarcoidosis and/or prognosing sarcoidosis, e.g., risk of progression to complicated sarcoidosis. In certain embodiments, the methods of the invention may be used in conjunction with any other diagnostic or prognostic criterion or method, including, but not limited to, currently known criterion or methods.

In certain embodiments, the method for determining whether a person has or is at risk of developing sarcoidosis includes detecting the presence or absence of a genetic variant of at least one of SESN3, NOG, FKBP1A, TSHZ2, RBM12B, or CX3CR1, the presence of the genetic variant indicating that the subject has or is at risk of developing the sarcoidosis.

In some embodiments, the method for determining whether a person has or is at risk of developing sarcoidosis includes detecting one or more SNPs selected from the SNPs listed in Table 5 (below). These SNPs may be detected alone or in combination with each other, i.e., the methods of the invention may include detection of from one to 30 of the SNPs listed in Table 5 in any possible combination. In certain embodiments, the method includes detecting the presence or absence of from one to 30 of the SNPs listed in Table 5 in any combination and detecting the presence or absence of any other SNP associated with a sarcoidosis or its prognosis.

Also provided is a method for testing for sarcoidosis or complicated sarcoidosis in a person that involves detecting the level of gene expression of one or more genes of the genes listed in Table 3, in any combination, in a sample from the person, a high level of HBEGF and/or SAP30 gene expression, and/or a low level of FITM2, TSHZ2, MEI1, LOC100287290, ZNF540, ZNF614, KIAA1147, LOC100132356, CX3CR1, RBM12B, FKBP1A, SERTAD1, APOBEC3D, KLRB1, CRIP1, NOG, SENS3, and/or ZNF671 gene expression in the person relative to a control being indicative of sarcoidosis and/or complicated sarcoidosis. The level of gene expression may be detected by measuring, directly or indirectly, HBEGF, SAP30, FITM2, TSHZ2, MEI1, LOCI 00287290, ZNF540, ZNF614, KIAA1147, LOCI 00132356, CX3CR1, RBM12B, FKBP1A, SERTAD1, APOBEC3D, KLRB1, CRIP1, NOG, SENS3, and/or ZNF671 mRNA or by measuring SAP30, FITM2, TSHZ2, MEI1, LOC100287290, ZNF540, ZNF614, KIAA1147, LOCI 00132356, CX3CR1, RBM12B, FKBP1A, SERTAD1, APOBEC3D, KLRB1, CRIP1, NOG, SENS3, and/or ZNF671 protein by any suitable method, several of which are known in the art. The control may include, for example, a sample from a person that does not have sarcoidosis or complicated sarcoidosis or a value or set of values, for example, a normal range, derived from several humans that do not have sarcoidosis. A high level of HBEGF or SAP30 gene expression relative to a control indicative of sarcoidosis and/or complicated sarcoidosis is a level that is 140% or more of the control. A low level of FITM2, TSHZ2, MEI1, LOC100287290, ZNF540, ZNF614, KIAA1147, LOCI 00132356, CX3CR1, RBM12B, FKBP1A, SERTAD1, APOBEC3D, KLRB1, CRIP1, NOG, SENS3, and/or ZNF671 gene expression relative to a control is a level that is 50% or less than that of the control.

The methods of the present invention are not limited to any particular way of detecting the presence or absence of a SNP or SNPs, and may employ any suitable method to detect the presence or absence of the SNP(s), of which numerous detection methods are known in the art.

In certain embodiments, the present invention provides a kit for predicting, diagnosing, or prognosing sarcoidosis in a person, the kit including at least one probe or primer for detecting the presence or absence of at least one genetic variation. In certain embodiments, the at least one genetic variation includes a genetic variant of at least one of SESN3, NOG, FKBP1A, TSHZ2, RBM12B, and CX3CR1. In certain embodiments, the kit includes at least one primer or probe for detecting more than one genetic variant of SESN3, NOG, FKBP1A, TSHZ2, RBM12B, and CX3CR1. In certain embodiments, the kit includes at least one probe or primer for detecting additional genetic variants diagnostic or predictive of risk for sarcoidosis. In some embodiments, the kit includes a probe or primer for detecting one or more SNPs selected from the SNPs listed in Table 5, either alone or in any possible combination.

Claims directed to kits for predicting, diagnosing, or prognosing sarcoidosis in a person “consisting essentially of” certain types of probes or primers is intended to capture kits that include probes or primers that are suitable primarily for detecting differential gene expression and/or genetic variants associated with sarcoidosis in humans as described herein, although the kits may also include additional probes or primers used as controls, for example, probes or primers for detecting “housekeeping” genes such β-actin, tubulin, or glyceraldehyde-3-phosphate dehydrogenase, for example. The use of the transitional phrase “consisting essentially of” is intended to exclude arrays, such as Affymetrix arrays, containing thousands of probes, the majority of which are unrelated to sarcoidosis. In certain embodiments, the kits may include buffers, enzymes, labels, and the like, for example, for use in isolating DNA or mRNA, generating cDNA, detecting level of gene expression of sarcoidosis-related genes, and/or detecting and/or sequencing specific sarcoidosis related genes and specific SNPs.

### Methods

### Subjects and PBMC Samples.

PBMC samples may be collected from subjects with sarcoidosis (n=39) and healthy controls (n=35) (Table I). The diagnosis of sarcoidosis was based on established joint international criteria (1). Subjects with other concurrent systemic inflammatory diseases were excluded. A total of 29 African descent American (AA) and 10 European descent American (EA) patients with sarcoidosis were included in the overall sarcoidosis cohort with 18 AA and 4 EA patients diagnosed with complicated sarcoidosis defined as cardiac sarcoidosis (e.g., ventricular arrhythmias), neurologic sarcoidosis (e.g., evidence of hyperdense MRI lesions), or severe pulmonary sarcoidosis (FVC<50%).

TABLE 1
Study subjects with racial and complication status.

| | Healthy | Uncomplicated | Complicated cases | | |
|---|---|---|---|---|---|
| Population | controls | cases | Cardiac | Neurologic | FVC <50% |
| AA | 8 | 11 | 5 | 5 | 10 |
| EA | 27 | 6 | 3 | 2 | 1 |
| Total | 35 | 17 | 8 | 7 | 11 |

Three individuals exhibited multiple complications; EA: European Americans (Caucasians); AA; African Americans.

### RNA Microarray Hybridization.

Total RNA was isolated from PBMCs using standard molecular biology protocols (n=74) without DNA contamination or RNA degradation. Sample processing (e.g., cDNA generation, fragmentation, end labeling, hybridization to Affymetrix GeneChip Human Exon 1.0 ST arrays) was performed by the University of Chicago Functional Genomics Facility per manufacturer's instructions.

### Microarray Data Preprocessing.

Expression arrays were analyzed using the Affymetrix Power Tools v.1.12.0 (http://www.affymetrix.com/). The experimental probe masking workflow provided by the Affymetrix Power Tools was utilized to filter the probeset (exon-level) intensity files by removing probes that contain known SNPs in the dbSNP database (2) (v129). Overall, of the ˜1.4 million probesets on the exon array, ˜350,000 probesets were found to contain at least one probe with a SNP (˜600,000 probes). The resulting probe signal intensities were quartile normalized over all 74 samples. Probeset expression signals were summarized with the robust multi-array average (RMA) algorithm (3) and log₂ transformed with a median polish. Expression signals of the −22,000 transcript clusters (gene-level) were then generated with the core set (i.e., with RefSeq-supported annotations) (4) of exons by taking averages of all annotated probesets for each transcript cluster. Adjustment for possible batch effect was conducted by COMBAT (http://jlab.byu.edu//ComB.\IJLI (5). A transcript cluster was considered to reliably expressed in these samples if the Affymetrix implemented DABG (detection above ground) (6) p-value was less than 0.0 I in at least 67% of the samples in each test group (healthy controls, patients with complicated sarcoidosis, patients with uncomplicated sarcoidosis) in each population, respectively. The analysis set was further limited to the genes with unique annotations (i.e., transcripts corresponding to unique genes) from the Affymetrix NetAffy website, accessed on Dec. 1, 2010). In total, 11,412 and 11,592 transcript clusters in the AA and EA samples, respectively, met these criteria and were further analyzed.

### Identification of Genes Differentially Expressed in Sarcoidosis and Complicated Sarcoidosis.

Genes on chromosomes X and Y were removed to avoid the potential confounding factor of gender. SAM (Significance Analysis of Microarrays) (7), implemented in the samr library of the R Statistical Package (8), was used to compare log₂-transformed gene expression levels between patients with complicated sarcoidosis and normal controls in the combined (AA and EA), EA, and AA samples, respectively. False discovery rate (FDR) was controlled using the q-value method (9). Transcripts with a fold-change greater than 1.4 and q-value less than 0.05 were deemed differentially expressed. Any enriched Kyoto Encyclopedia of Genes and Genomes (KECJG) (10) physiological pathways among the differential genes relative to the final analysis set was searched using the NIH/DAVID (11, 12). An adjusted p-value <0.05 after the Benjamini-Horchberg procedure (13) was used as the cutoff.

### Identification of Gene Signature for Classifying Sarcoidosis and Complicated Sarcoidosis.

To identify gene signatures useful in the diagnosis and classification of sarcoidosis, a machine learning algorithm based on support vector machine (SVM) using a linear kernel, was applied in combination with recursive feature elimination (RFE) for generating a predictive model (14-17). The e1071 library of the R Statistical Package (8) was used to conduct SVM and RPE. In each round of RFE, the SVM linear classifier was trained by the pooled samples from both AA and EA, including all the healthy controls and sarcoidosis patients. The gene signature that was comprised of the smallest number of genes with significant peak prediction accuracy was used in subsequent analyses. To test the performance of the gene signature, 1,000 times of five-fold cross-validation was conducted using SVM. In addition, the gene signature was also tested for classification accuracy in AA and EA samples, separately.

Genotypic Data on SNPs Residing within Sarcoidosis Signature Genes.

Genotypic data for signature gene SNPs was obtained via analysis of a sarcoidosis GWAS (genome-wide association study) with current SNP and gene annotations obtained from the Affymetrix NetAffy website (accessed on Dec. 1, 2010). The sarcoidosis GWAS dataset was comprised of 195 (46 complicated) EA cases and 212 (68 complicated) AA cases with SNPs genotyped using the Affymetrix 6.0 SNP Array. Briefly, the SNPRMA and CRLMM packages of the Bioconductor Project (18) were used to preprocess the scanned intensities and genotype calling. Genotypic data were checked for genotyping rate and Hardy-Weinberg Equilibrium (P<10⁻⁶) and publicly available dbGaP (http://www.ncbi.nlm.nih.gov/gap) data for the GAIN Genome-wide Association Study of Schizophrenia (v3, October, 2010) utilized as healthy normal controls. Specifically, 1-1 matched dbGaP samples were selected based on general genetic background (i.e., according to the weighted distance between each case and controls from a principal component analysis on common SNPs with minor allele frequency (MAF) greater than 0.05 in normal individuals) and gender for each population. The allele frequencies of common SNPs (MAF>0.05) in signature genes and genes in candidate pathways were compared using PLINK (19) between patients and normal controls, as well as between complicated and uncomplicated sarcoidosis patients in each population, separately. Since this is a targeted analysis on a small number of signature and candidate genes, a cutoff of nominal p-value <0.01 was chosen to call significant relationships.

### Generation of a T Cell Receptor (TCR)IJAK-STAT/Cytokine-Cytokine Receptor Signaling Pathway Gene Signature.

T cell receptor (TCR) signaling pathway genes, as annotated by the KEGG (10), are comprised of the TCR and co-stimulatory molecules such as CD28 and IL7R, a gene highly expressed in both naive and memory T cells and implicated in sarcoidosis susceptibility (20-22). Because the JAK-STAT (JS) and cytokine-cytokine receptor (CCR) signaling pathways are implicated in sarcoidosis pathogenesis, genes within these two pathways were also collected from KEGG (10). TCR/JS/CCR signaling pathway genes differentially expressed between EA or AA patients with complicated sarcoidosis and normal controls were estimated for their power to classify sarcoidosis cases and normal controls, as well as complicated and uncomplicated sarcoidosis in the combined (EA and AA), EA, and AA samples, separately. Using linear SVM, a five-fold cross-validation (repeated for 1,000 times) of the predictive models based on TCR/JS/CCR signaling pathway genes was performed. The means of the predictive accuracy of the TCR/JS/CCR signaling pathway genes were compared with those of a 20-gene signature by standard t test (P<0.05 as the cutoff for significance).

### Results

### Patient Characteristics.

The clinical characteristics of study patients are displayed in Table 2. Significant differences in age, gender, race and pulmonary function studies did not exist between uncomplicated and complicated sarcoidosis cases (P>0.05 by χ² test for gender and p>0.05 by t-test for the other characteristics). Uncomplicated sarcoidosis cases trended toward higher corticosteroid usage whereas complicated sarcoidosis cases trended toward higher methotrexate usage and were more likely to be receiving anti-TNFα. therapy. However, these differences were not statistically significant (P>0.05 for all drugs) (Table 2). Predictably, complicated pulmonary sarcoidosis cases exhibited significantly reduced pulmonary function compared to the other study groups (data not shown).

TABLE 2
Patient characteristics and concomitant medications.

| | Uncomplicated | Complicated |
|---|---|---|
| Characteristics | sarcoidosis (n = 17) | sarcoidosis (n = 22) |
| Age | 49 ± 10 | 47 ± 9 |
| Gender (Male/Female) | 5/12 | 5/17 |
| FVC, L | 2.9 ± 0.8 | 2.7 ± 1.5 |
| FVC, percent of predicted | 74 ± 17 | 65 ± 31 |
| FEV₁, L | 2.2 ± 0.7 | 2.1 ± 1 |
| FEV1, percent of predicted | 74 ± 17 | 67 ± 30 |
| DL_{CO}, percent of predicted | 74 ± 23 | 65 ± 28 |
| Corticosteroids, n (dose, mg prednisone equivalent per day) | 7(20 ± 16) | 11(13 ± 11) |
| Methotrexate, n (dose, mg per week) | 3(12.25 ± 3.5) | 7(11 ± 4) |
| Mycophenolate, n (dose, mg per day) | 1(250) | 3(667 ± 289) |
| Anti-TNF alpha therapy, n | 0 | 3 |

### Identification of Differentially-Expressed Genes in Sarcoidosis.

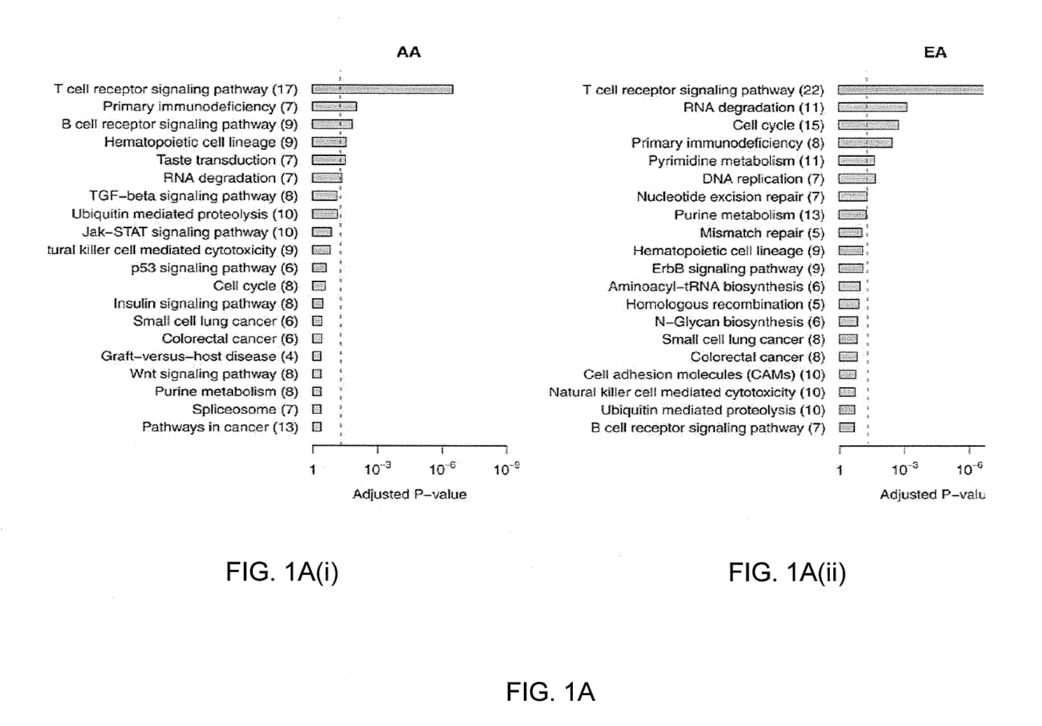

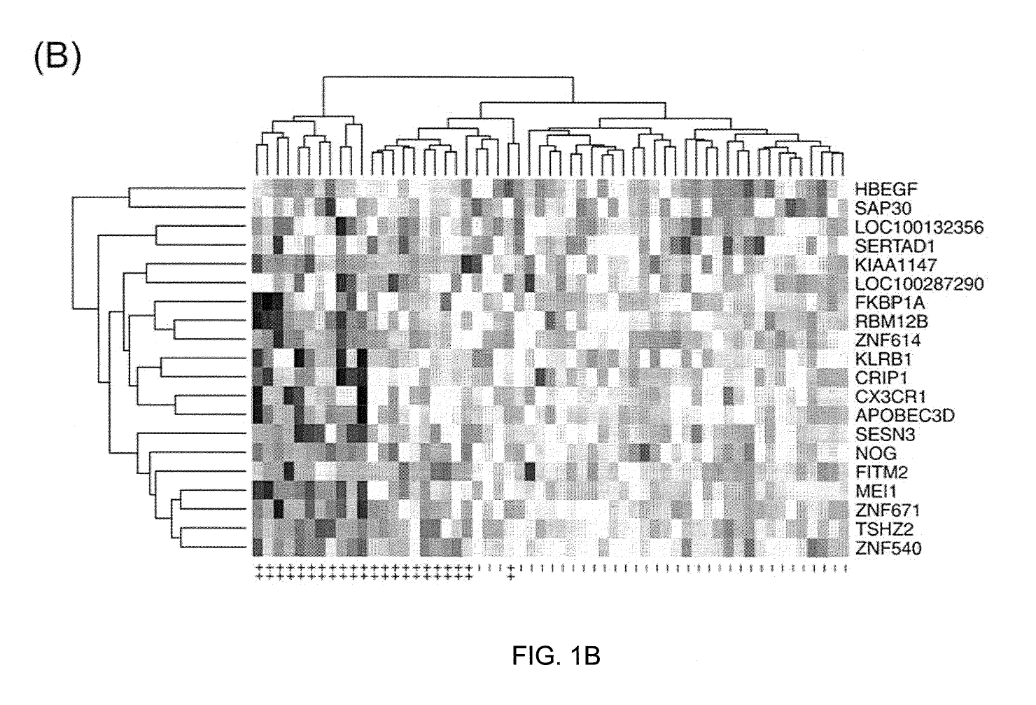

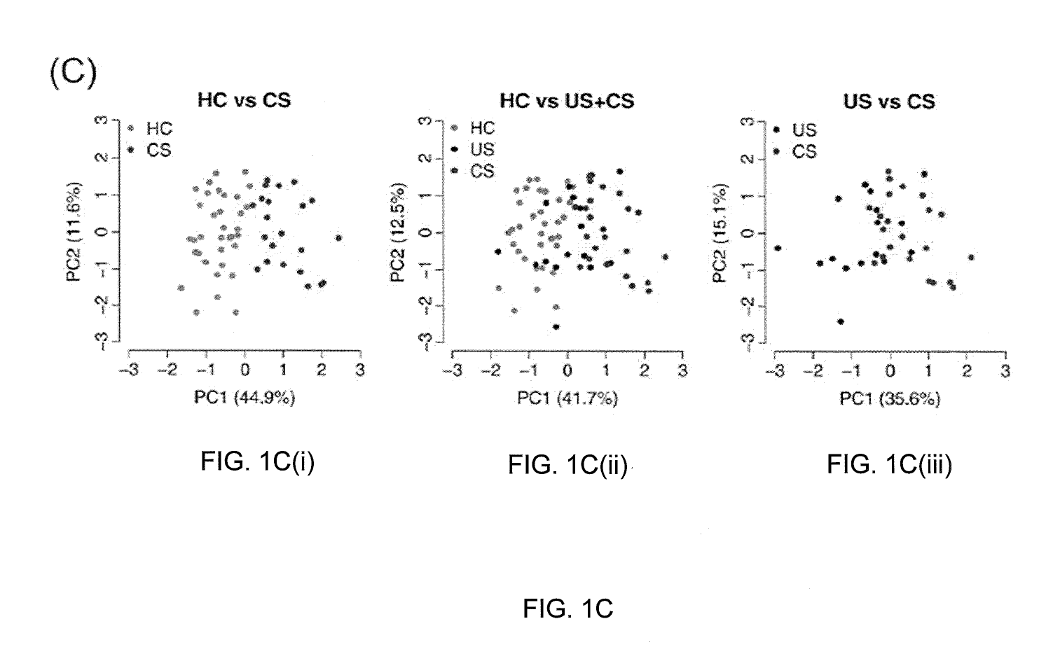

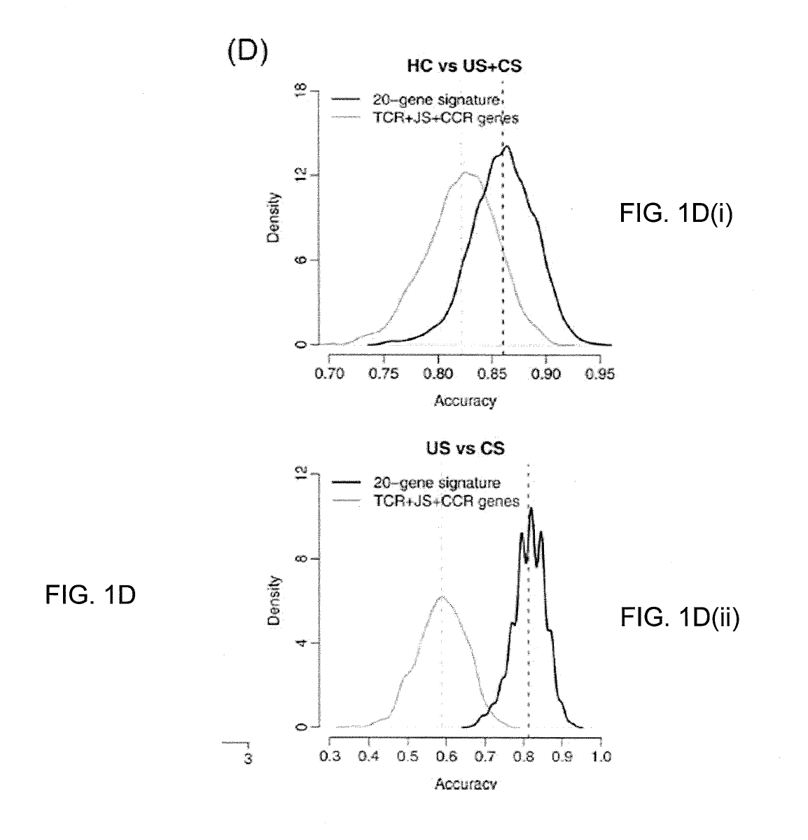

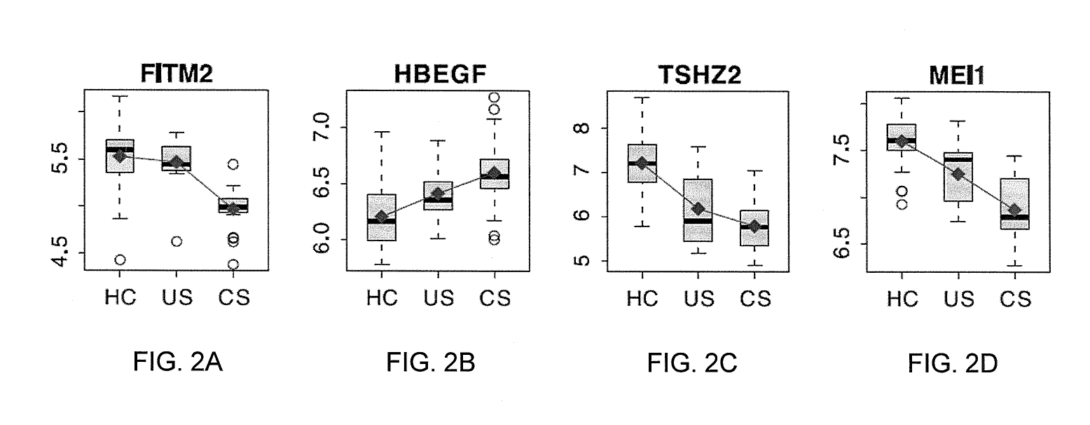

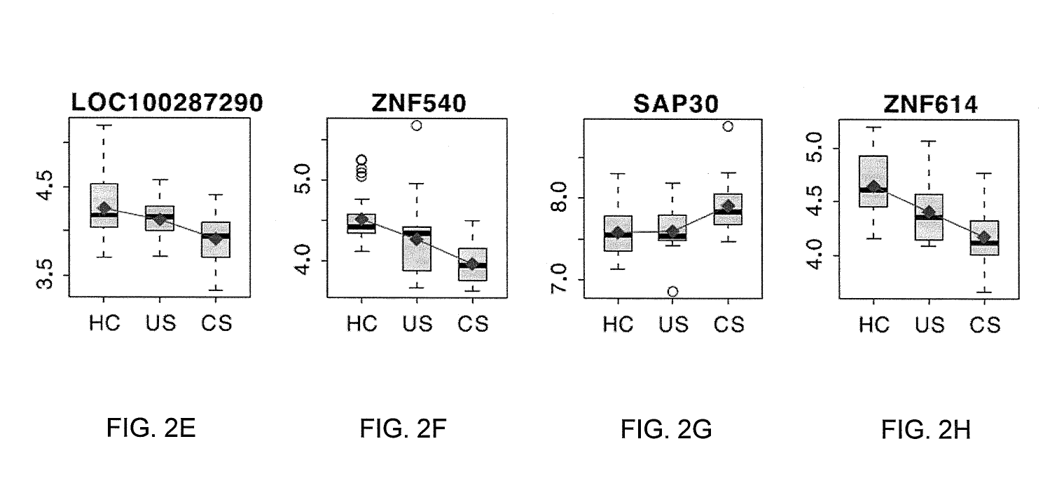

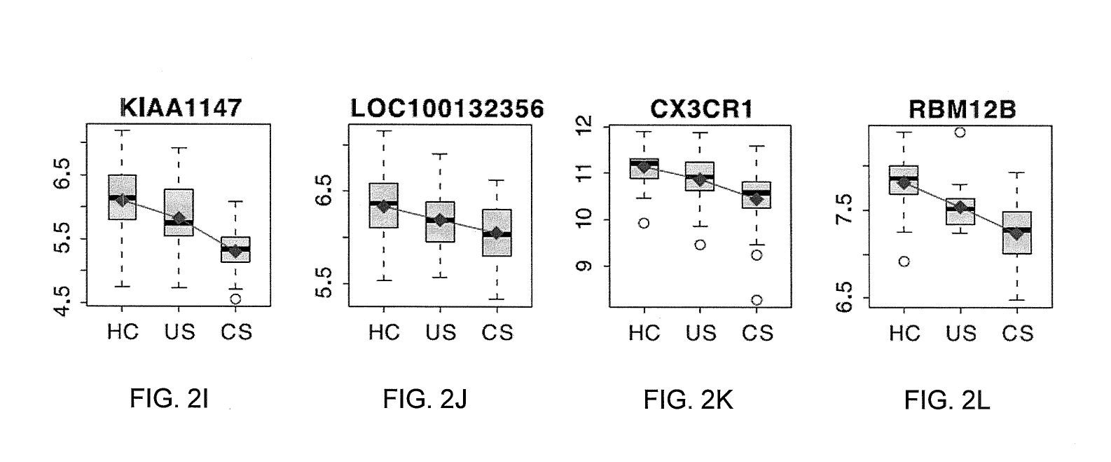

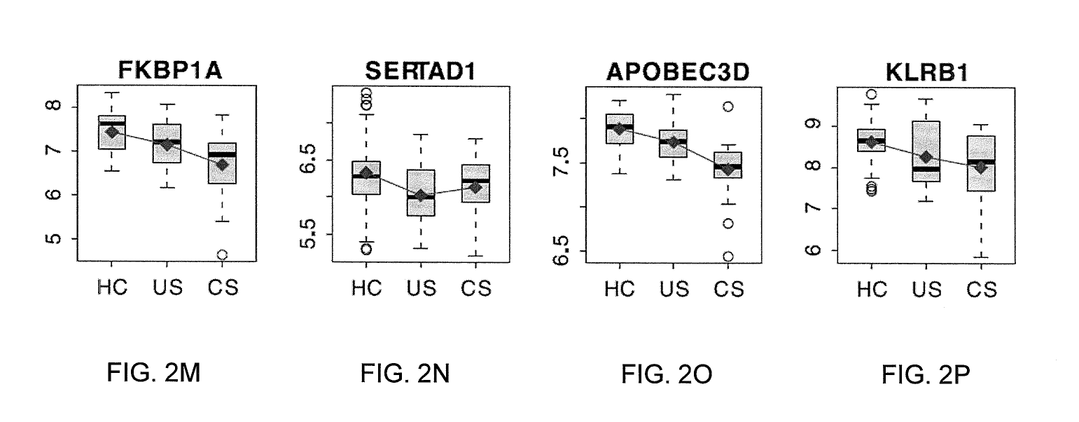

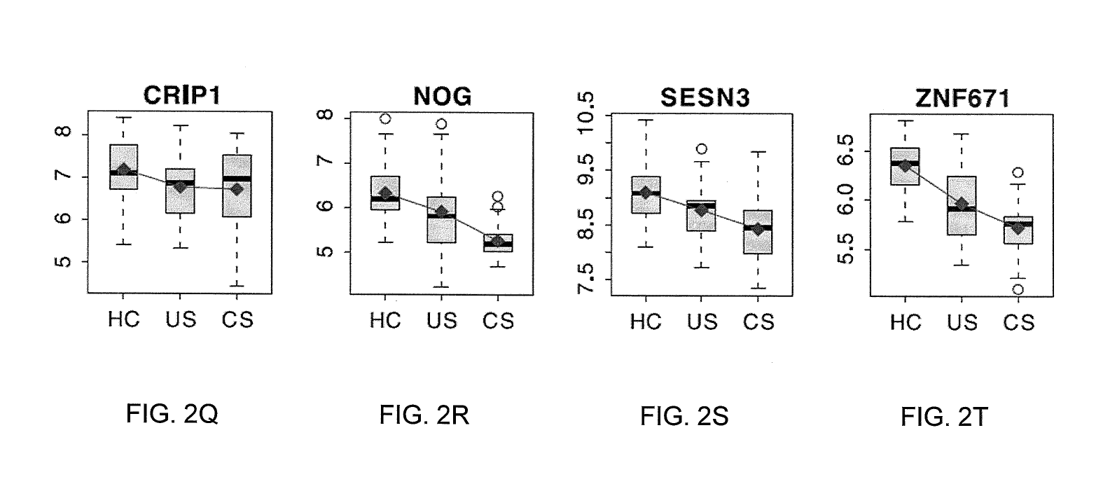

All cases with diagnoses of cardiac, neurologic, or progressive pulmonary sarcoidosis (FVC<50%) comprised the cohort labeled as ‘complicated sarcoidosis’. At the specified significance level (fold-change>1.4, q-value <0.05), 316 genes were differentially expressed between all sarcoidosis cases and healthy controls in the combined samples (pooled AAs and EAs). For individual populations, 118 genes were differentially-expressed between all AA cases and controls, whereas 861 genes were differentially expressed between all EA cases and controls. In contrast, 1124 genes were differentially expressed between complicated sarcoidosis cases and healthy controls in the combined samples. For individual population, 730 and 980 genes were differentially expressed between AA and EA cases with complicated sarcoidosis and healthy controls, respectively with the TCR. Signaling pathway significantly enriched among complicated sarcoidosis-associated genes in both populations (adjusted P<0.05) (FIG. 1A).

Identifying a gene signature for complicated sarcoidosis. To identify a universal gene signature for complicated sarcoidosis in both AA and EA populations, an initial analysis set comprised of 1233 genes differentially expressed between AA or EA complicated sarcoidosis cases vs. healthy controls was utilized for the SVM algorithm. A 20-gene signature (Table 3) was chosen as the most parsimonious signature with the peak prediction accuracy and accurately distinguished patients with complicated sarcoidosis from healthy controls (FIGS. 1B and 1C), or from uncomplicated sarcoidosis (FIG. 1C). Two genes within the unbiased 20-gene signature, HJJEGF (heparin-binding EGF-like growth factor) and SAP30 (Sin3A-associated protein, 30 kDa), were strongly up-regulated in complicated sarcoidosis whereas the remaining 18 signature genes were down-regulated in complicated sarcoidosis (Supplemental FIG. 1). The non-targeted 20-gene signature distinguished all sarcoidosis patients from healthy controls with an accuracy of 86.0% (sensitivity=88.2% and specificity=83.3%) in the combined samples (pooled AAs and EAs) (FIG. 1D). The discriminative accuracy became 88.2% and 94.2% in separating sarcoidosis cases from healthy controls in AA and EA, respectively. When distinguishing complicated sarcoidosis cases from uncomplicated sarcoidosis cases, the accuracy was 81.4% (sensitivity=87.0% and specificity=74.2%) in the combined samples (FIG. 1D) but was reduced to 83.7% and 64.5% in separating complicated sarcoidosis cases from uncomplicated sarcoidosis cases in AA and EA, respectively.

TABLE 3
The unbiased 20-gene signature for complicated sarcoidosis.

| Gene symbol | Gene title | Weight |
|---|---|---|
| FITM2 | fat storage-inducing transmembrane protein 2 | 0.04872 |
| HBEGF | heparin-binding EGF-like growth factor | 0.04791 |
| TSHZ2 | teashirt zinc finger homeobox 2 | 0.04648 |
| MEI1 | meiosis inhibitor 1 | 0.04218 |
| LOC100287290 | cytokine receptor CRL2 | 0.03851 |
| ZNF540 | zinc finger protein 540 | 0.03776 |
| SAP30 | Sin3A-associated protein, 30kDa | 0.02935 |
| ZNF614 | zinc finger protein 614 | 0.02715 |
| KIAA1147 | KIAA1147 | 0.02585 |
| LOC100132356 | hypothetical protein LOC100132356 | 0.02561 |
| CX3CR1 | chemokine (C-X3-C motif) receptor 1 | 0.02547 |
| RBM12B | RNA binding motif protein 12B | 0.02286 |
| FKBP1A | FK506 binding protein 1A, 12kDa | 0.02157 |
| SERTAD1 | SERTA domain containing 1 | 0.02119 |
| APOBEC3D | apolipoprotein B mRNA editing enzyme, catalytic polypeptide-like 3D | 0.02106 |
| KLRB1 | killer cell lectin-like receptor subfamily B, member 1 | 0.01979 |
| CRIP1 | cysteine-rich protein 1 (intestinal) | 0.01889 |
| NOG | noggin | 0.01724 |
| SESN3 | sestrin 3 | 0.01701 |
| ZNF671 | zinc finger protein 671 | 0.01657 |

Here, the weight of each gene represents the frequency of the gene being selected during the last round of RFE procedure.

### Evaluation of a Sarcoidosis-Related TCR/JS/CCR Signaling Pathway Gene Signature.

As the T cell receptor pathway (TCR), the JAK STAT signaling pathway (JS) and the cytokine-cytokine receptor signaling pathway (CCR) have all been implicated in sarcoidosis, a 31 gene signature comprised of TCR/JS/CCR signaling pathway genes implicated associated with sarcoidosis was assessed as a potential molecular biomarker in identifying cases or risk for complicated sarcoidosis (Table 4). Overall, this TCR/JS/CCR signaling pathway signature differentiated sarcoidosis from healthy controls with a prediction accuracy of 82.2% (FIG. 1D), but exhibited a substantially reduced prediction accuracy of <60% in distinguishing complicated sarcoidosis from uncomplicated sarcoidosis (FIG. 1D). The discriminative accuracy of this TCR/JS/CCR signature was 83.2% in separating all AA sarcoidosis patients from healthy controls but only 69.7% for distinguishing AA complicated sarcoidosis cases from uncomplicated sarcoidosis. Similarly, in EA cases, the accuracy of the TCR/JS/CCR signature was 75.1% for distinguishing sarcoidosis patients from healthy controls, but only 37.5% in distinguishing EA patients with complicated sarcoidosis from uncomplicated EA sarcoidosis cases. Comparison of the prediction accuracy in both the TCR/JS/CCR and unbiased 20-gene signatures in combined EA and AA cases (FIG. 1D) revealed the superior performance of the unbiased 20-gene sarcoidosis signature (P<10⁻¹⁵ by t-test).

TABLE 4
The 31 differentially-expressed TCR/JS/CRR signaling pathway genes in sarcoidosis.

| | | AA | | EA | |
|---|---|---|---|---|---|
| | | Fold | FDR | Fold | FDR |
| Gene symbol | Gene title | change | (%) | change | (%) |
| CD247 | CD247 molecule | 0.63 | 0.0 | 0.62 | 0.2 |
| CD28 | CD28 molecule | 0.60 | 0.3 | 0.55 | 0.5 |
| CD3D | CD3d molecule, delta (CD3-TCR complex) | 0.52 | 0.2 | 0.44 | 0.0 |
| CD3E | CD3e molecule, epsilon (CD3-TCR complex) | 0.67 | 1.0 | 0.49 | 0.2 |
| CD3G | CD3g molecule, gamma (CD3-TCR complex) | 0.52 | 0.2 | 0.45 | 0.0 |
| CD8A | CD8a molecule | 0.84 | 7.4 | 0.63 | 0.8 |
| CBLB | Cas-Br-M (murine) ecotropic retroviral transforming | 0.68 | 0.5 | 0.65 | 0.2 |
| | sequence b | | | | |
| GRAP2 | GRB2-related adaptor protein 2 | 0.84 | 7.4 | 0.71 | 1.2 |
| ITK | IL2-inducible T-cell kinase | 0.52 | 0.2 | 0.42 | 0.0 |
| NCK1 | NCK adaptor protein 1 | 0.88 | 11.3 | 0.71 | 0.2 |
| RASGRP1 | RAS guanyl releasing protein 1 (calcium and DAG- | 0.51 | 0.0 | 0.48 | 0.0 |
| | regulated) | | | | |
| DLG1 | discs, large homolog 1 (Drosophila) | 0.73 | 1.5 | 0.69 | 0.8 |
| ICOS | inducible T-cell co-stimulator | 0.59 | 0.2 | 0.61 | 1.7 |
| IFNG | interferon, gamma | 0.56 | 0.0 | 0.74 | 4.7 |
| IL7R | interleukin 7 receptor | 0.69 | 3.4 | 0.51 | 0.5 |
| JUN | jun oncogene | 0.67 | 4.3 | 2.17 | 23.7 |
| LCK | lymphocyte-specific protein tyrosine kinase | 0.70 | 0.5 | 0.61 | 0.2 |
| MAPK9 | mitogen-activated protein kinase 9 | 0.78 | 3.4 | 0.71 | 0.5 |
| MALT1 | mucosa associated lymphoid tissue lymphoma | 0.66 | 0.3 | 0.69 | 0.5 |
| | translocation gene 1 | | | | |
| NFATC2 | nuclear factor of activated T-cells, cytoplasmic, | 0.59 | 0.0 | 0.57 | 0.0 |
| | calcineurin-dependent 2 | | | | |
| NFATC3 | nuclear factor of activated T-cells, cytoplasmic, | 0.66 | 0.0 | 0.71 | 0.2 |
| | calcineurin-dependent 3 | | | | |
| PIK3CA | phosphoinositide-3-kinase, catalytic, | 0.76 | 2.3 | 0.69 | 0.5 |
| | alpha polypeptide | | | | |
| PLCG1 | phospholipase C, gamma 1 | 0.60 | 0.0 | 0.62 | 0.2 |
| AKT3 | v-akt murine thymoma viral oncogene homolog 3 | 0.60 | 0.3 | 0.54 | 0.0 |
| | (protein kinase B, gamma) | | | | |
| ZAP70 | zeta-chain (TCR) associated protein kinase 70kDa | 0.72 | 0.5 | 0.66 | 0.5 |
| CCND2 | cyclin D2 | 0.62 | 0.3 | 0.51 | 0.0 |
| IL2RA | interleukin 2 receptor, alpha | 0.61 | 0.3 | 0.78 | 17.2 |
| IL2RB | interleukin 2 receptor, beta | 0.66 | 0.3 | 0.64 | 1.7 |
| STAT4 | signal transducer and activator of transcription 4 | 0.61 | 0.3 | 0.52 | 0.0 |
| SPRED1 | sprouty-related, EVH1 domain containing 1 | 0.60 | 0.0 | 0.77 | 4.7 |
| SOCS4 | suppressor of cytokine signaling 4 | 0.71 | 0.3 | 0.77 | 0.8 |

EA: Caucasian Americans; AA: African Americans; FDR: false discovery rate.

Finally, as sarcoidosis and IPF represent the most common interstitial lung diseases (ILDs) of unknown etiology, the capacity for the unbiased 20-gene and TCR/JS/CCR sarcoidosis gene signatures to distinguish sarcoidosis cases from IPF cases (n=46) was assessed. Each signature performed with comparable prediction accuracy in IPF and sarcoidosis with the 20-gene signature (77.2%) slightly superior to the TCR/JS/CCR signaling pathway signature (76.5%) in distinguishing sarcoidosis from IPF cases (P<10⁻⁵ by t-test).

### Use of Genetic Variants to Validate Sarcoidosis Gene Signatures.

A genome-wide association study (GWAS) (Affymetrix 6.0 SNP array) involving 407 sarcoidosis cases including 212 AAs (including 68 complicated cases) and 195 EAs (including 46 complicated cases) was performed and allele frequencies of 1,300 common SNPs residing in unbiased sarcoidosis signature genes analyzed in sarcoidosis cases and healthy controls. At the nominal P-value <0.01, 30 SNPs from 6 unbiased 20-gene signature genes were found to be significantly associated with sarcoidosis (Table 5), including 4 genes which overlapped between the AA and EA samples (NOG [noggin], RMB12B [RNA binding motif protein 12B], SESN3 [sestrin 3], TSHZ2 [teashirt zinc finger homeobox 2]). The most highly significant signature gene SNP in AAs was rs629508 (P=1.7×10⁻³) in SESN3, whereas in EA cases, the most significant SNP was rs2618134 (P 4.7×10⁻⁵) in RBMI2B. Several SNPs were also significantly associated with complicated sarcoidosis, including rs629508 (P=5.4×10⁻⁵) and rs1294689 (P=3.6×10⁻⁵) in the AA samples and rs10485815 (P=2.8×10⁻⁵) in the EA samples (Table 5). In comparison, from ˜3,800 common SNPs residing in TCR/JS/CCR signature genes, 37 SNPs were associated with sarcoidosis in AA samples, whereas 34 SNPs were significant in EA samples, respectively. The most highly significant TCR-JS-CCR signature gene SNP in Ms was rs2131817 (P<1.4×10⁻⁵) in AK3, whereas in EA cases, the most significant SNP was rs7614488 (P=7.8×10⁻⁷) in CBLB. Several TCR/JS/CCR signature gene SNPs, rs2953040 and rs6791765 in CBL/3 (Cas-Br-M, murine, ecotropic retroviral transforming sequence b) and rs2131817 in AKT3 were significantly associated with sarcoidosis in both EA and AA sarcoidosis cases (P<0.01).

TABLE 5
SNPs significantly associated with sarcoidosis within the unblased 20 signature genes
(P < 0.01).

| | | | | | | | Complicated | |
|---|---|---|---|---|---|---|---|---|
| | | | | | Sarcoidosis | | sarcoidosis | |
| | | | | | vs | | vs | |
| | | | | | healthy | | uncomplicated | |
| | SNP | dbSNP | Gene | Gene | controls | | sarcoidosis | |
| Population | chromosome | RS ID | symbol | relationship | P | OR | P | OR |
| African | 11 | rs629508 | SESN3 | intron | 1.7E−03 | 1.645 | 5.4E−05 | 0.254 |
| Americans | 17 | rs7219027 | NOG | downstream | 4.3E−03 | 1.487 | | |
| | 20 | rs1294689 | FKBP1A | intron | 4.8E−03 | 1.536 | 3.6E−05 | 2.710 |
| | 11 | rs12280779 | SESN3 | upstream | 5.5E−03 | 1.555 | | |
| | 20 | rs201812 | TSHZ2 | intron | 7.5E−03 | 1.438 | | |
| | 8 | rs16914980 | RBM12B | downstream | 7.8E−03 | 0.475 | | |
| | 8 | rs491546 | RBM12B | downstream | 8.9E−03 | 0.529 | | |
| | 8 | rs7821394 | RBM12B | downstream | 9.7E−03 | 0.728 | | |
| European | 8 | rs2618134 | RBM12B | downstream | 4.7E−05 | 2.183 | | |
| Americans | 8 | rs6993453 | RBM12B | downstream | 3.1E−04 | 1.819 | | |
| | 20 | rs1293381 | TSHZ2 | intron | 3.8E−04 | 0.614 | | |
| | 8 | rs2595613 | RBM12B | downstream | 4.3E−04 | 2.357 | | |
| | 8 | rs12544183 | RBM12B | downstream | 5.3E−04 | 1.916 | | |
| | 17 | rs1914986 | NOG | downstream | 9.6E−04 | 1.946 | | |
| | 8 | rs279959 | RBM12B | downstream | 1.2E−03 | 1.621 | | |
| | 3 | rs4676483 | CX3CR1 | downstream | 1.7E−03 | 1.671 | | |
| | 8 | rs10808648 | RBM12B | downstream | 2.0E−03 | 1.540 | | |
| | 20 | rs1326861 | TSHZ2 | downstream | 2.1E−03 | 1.469 | | |
| | 11 | rs11021203 | SESN3 | upstream | 4.0E−03 | 0.570 | | |
| | 11 | rs16922328 | SESN3 | upstream | 5.5E−03 | 1.599 | | |
| | 20 | rs6068555 | TSHZ2 | intron | 6.1E−03 | 0.710 | | |
| | 8 | rs549043 | RBM12B | downstream | 6.2E−03 | 1.805 | | |
| | 8 | rs566469 | RBM12B | downstream | 6.4E−03 | 1.718 | | |
| | 20 | rs6097326 | TSHZ2 | intron | 6.4E−03 | 0.569 | | |
| | 20 | rs6068566 | TSHZ2 | downstream | 6.6E−03 | 0.706 | | |
| | 3 | rs6773586 | CX3CR1 | upstream | 8.3E−03 | 0.560 | | |
| | 8 | rs278586 | RBM12B | downstream | 9.0E−03 | 1.821 | | |
| | 8 | rs7829923 | RBM12B | downstream | 9.2E−03 | 1.434 | | |
| | 17 | rs17820808 | NOG | downstream | 9.3E−03 | 0.471 | | |
| | 20 | rs10485815 | TSHZ2 | intron | 9.8E−03 | 1.653 | 2.8E−05 | 3.535 |

GWAS results between complicated and uncomplicated sarcoidosis were listed only for the SNPs with P < 0.01,
OR: odds ratio.

As described above, universal and racially-specific gene signatures are novel biomarkers for the presence of sarcoidosis as well as for the presence and/or susceptibility of the development of complicated sarcoidosis were identified. Leveraging whole genome expression profiles in a cohort of sarcoidosis patients, an unbiased gene signature comprised of 20 autosomal genes was identified which distinguished sarcoidosis cases from healthy individuals and, importantly, differentiated patients with complicated sarcoidosis from patients with uncomplicated sarcoidosis. The 20-gene signature exhibited equivalent prediction accuracy to other sarcoidosis signatures containing a greater number of genes (such as 39-gene and 78-gene sarcoidosis signatures) with each signature superior in accuracy to signatures with fewer genes (e.g., the 10 gene signature). The expression levels of the majority of these 20 signature genes showed a pattern of an additive model between uncomplicated and complicated sarcoidosis, i.e., when the signature gene is up-regulated, patients with complicated sarcoidosis exhibited higher expression levels than patients with uncomplicated sarcoidosis. Conversely, complicated sarcoidosis cases exhibit lower expression levels than patients with uncomplicated sarcoidosis when the signature gene is down-regulated. In the sarcoidosis signature, 19 of 20 genes performed unidirectionally (up-regulation or down-regulation) in both complicated and uncomplicated sarcoidosis. Therefore, the 20-gene signature appears to not only capture differences between complicated sarcoidosis and healthy controls, but potentially conveys information regarding differences between sarcoidosis cases (both complicated and uncomplicated) and healthy controls.

Gene products encoded by TCR/JS/CCR signaling pathway genes have been implicated in sarcoidosis pathogenesis (8, 54) and these signature genes were enriched among the differential genes between EA and AA cases with complicated sarcoidosis cases and healthy controls. The utility of a TCR/JS/CCR signaling pathway gene signature in classifying sarcoidosis cases was compared to the unbiased 20-gene signature. Both signatures performed with high level prediction accuracy (>80%) in distinguishing cases with sarcoidosis from healthy controls. In contrast, the prediction accuracy of the 20-gene signature was much superior to the TCR/JS/CCR signaling pathway gene signature in classifying combined AA and EA patients with complicated and uncomplicated sarcoidosis (81.4% vs. 58.8%, P<10⁻¹⁵, t-test). The unbiased nature of the 20-gene signature may allow better capture of the characteristics of complicated sarcoidosis compared to the more restrictive TCR/JS/CCR signaling pathway signature genes. The potential role of TCR/JS/CCR signaling pathways genes in the development of sarcoidosis was confirmed by the capacity of this signature to successfully differentiate the majority of sarcoidosis and healthy controls. However, either sarcoidosis disease progression or the development of complicated sarcoidosis likely requires the participation of genes and pathways extending beyond the TCR/JS/CCR pathway. These findings underscore the complex pathobiology of this disorder and implicate the necessity of global and unbiased approaches.

The classification accuracy of the 20-gene sarcoidosis signature was further evaluated separately in EA and AA samples and it was discovered that the 20-gene signature demonstrates >85% accuracy for classifying either EA or AA sarcoidosis cases (complicated and uncomplicated) from healthy controls. In contrast, the 20-gene sarcoidosis signature differentiated complicated sarcoidosis and uncomplicated sarcoidosis cases with an accuracy >80% in AA cases, but only ˜60% in EA cases, potentially the relative smaller complicated EA sample size or a bias for AA expression dysregulation driven by greater genetic variation, an issue which requires further examination. Both the 20-gene signature and TCR/JS/CCR-gene signature successfully discriminated sarcoidosis cases from IPF patients with similar prediction accuracies reflecting the differences in immunopathogenesis, clinical course, prognosis, and response to steroid treatment in these two fibrotic lung disorders. From this finding, additional clinical utility of the signature as a diagnostic biomarker for sarcoidosis may be inferred.

The 20-gene signature is comprised of novel candidate genes in diagnosing sarcoidosis susceptibility and prognosing severity. As a complementary method to validate these findings, allele frequencies of both unbiased 20-gene sarcoidosis signature SNPs as well as TCR/JS/CCR signaling pathway signature gene SNPs were examined in sarcoidosis cases and healthy controls embedded within a GWAS dataset constructed by genome-wide assessment of genetic variants in over 400 EA and AAs with sarcoidosis. As genetic variants, such as SNPs and copy number variants (CNVs), contribute significantly to variations in gene expression, SNPs were annotated to the genomic regions of these signature genes (based on the Affymetrix annotation) and, therefore, potentially contribute to gene expression variation by acting as cis-eQTLs. From 1,300 SNPs in the 20 signature genes, 30 SNPs (corresponding to 6 signature genes) were identified that are significantly associated with sarcoidosis in either EA or AA samples, suggesting a potential role of these cis-acting SNPs in regulating the expression of sarcoidosis signature genes. Similarly, from 3,800 SNPs in TCR/JS/CCR signature genes, relationships between SNPs and sarcoidosis were observed. The results suggest that genetic variants via cis-acting eQTLs may contribute to the variation in expression of sarcoidosis signature genes. It is further recognized that additional factors, such as trans-acting eQTLs, environmental factors, or epigenetic pathways, may contribute substantially to signature gene expression variation. Further investigations involving genome-wide genotypic data (e.g., for mapping trans-acting eQTLs) and expression data on the same samples could potentially provide greater insights into the contribution of genetics to the identified gene signature.

Quantitative abnormalities in T cells have been described in the peripheral blood of patients with sarcoidosis with significant lymphopenia, involving CD4, CDS, and CD 19 positive cells, common in sarcoidosis patients and correlating with disease severity. Individual signatures genes may not only have a role in the pathophysiology of sarcoidosis but could be potentially approached as novel therapeutic targets for the disease. For example, 11/VJEGF, a member of the EGF family of growth factors, is a potent mitogen and chemoattractant for many cell types including fibroblasts, smooth muscle cells and epithelial cells. A substantial body of evidence suggests that HBGEF plays a role in wound healing and response to injury leading to speculation that HBEGF may represent a target involved in the pathobiology of chronic lung sarcoidosis and a novel therapeutic target.

Recently, lung gene expression profiles were compared between patients with self-limiting sarcoidosis and those with progressive restrictive fibrotic disease with a greater number of down-regulated genes versus up-regulated genes identified in patients with progressive pulmonary sarcoidosis. These findings are highly consistent with the expression profile of the 20-gene signature in patients with complicated sarcoidosis. No overlap between sarcoidosis signature genes and the differentially expressed genes produced by comparison of self-limited and progressive lung sarcoidosis was identified. The lack of overlap may reflect greater severity of disease in the cohort with cardiac and neurologic sarcoidosis in addition to cases with severe lung disease. In addition, the studies did not involve lung tissue expression but rather analysis of PBMCs and therefore tissue-specific expression may also contribute to this lack of overlap.

In summary, an unbiased 20-gene molecular gene signature has been identified as a novel molecular biomarker in the diagnosis of sarcoidosis and complicated sarcoidosis with substantial accuracy in both EA and AA sarcoid cases.

While the disclosure is susceptible to various modifications and alternative forms, specific exemplary embodiments of the present invention have been shown by way of example in the drawings and have been described in detail. It should be understood, however, that there is no intent to limit the disclosure to the particular embodiments disclosed, but on the contrary, the intention is to cover all modifications, equivalents, and alternatives falling within the scope of the disclosure as defined by the appended claims.

What is claimed is:

1. A kit for assessing risk of a person developing sarcoidosis and/or complicated sarcoidosis, the kit comprising a set of probes consisting essentially of probes for detecting expression levels of one or more genes listed in Table 3 and/or the presence or absence of one or more single polynucleotide polymorphism listed Table 5.

2. The kit of claim 1, wherein the set of probes consists essentially of probes for detecting expression levels of one or more genes listed in Table 3.

3. The kit of claim 1, wherein the set of probes consists essentially of probes for detecting expression levels of one or more genes listed in Table 3.

4. The kit of claim 3, wherein the set of probes consists essentially of probes for detecting expression levels of one or more genes selected from SAP30, CX3CR1, FKBP1A, SERTAD1, APOBEC3D, KLRB1, CR1P1, NOG, SESN3, and ZNF671.

5. The kit of claim 4, wherein the set of probes consists essentially of probes for detecting expression levels of from two to ten genes selected from SAP30, CX3CR1, FKBP1A, SERTAD1, APOBEC3D, KLRB1, CR1P1, NOG, SESN3, and ZNF671.

6. The kit of claim 5, wherein the set of probes consists essentially of probes for detecting expression levels of SAP30, CX3CR1, FKBP1A, SERTAD1, APOBEC3D, KLRB1, CR1P1, NOG, SESN3, and ZNF671.

7. The kit of claim 4, wherein the set of probes further comprises probes for detecting expression levels of at least one of HBEGF, FITM2, TSHZ2, MEI1, LOC100287290, ZNF540, ZNF614, KIAA1147, LOC100132356, and RMB12B.

8. The kit of claim 7, wherein the set of probes consists essentially of probes for detecting expression levels of SAP30, CX3CR1, FKBP1A, SERTAD1, APOBEC3D, KLRB1, CR1P1, NOG, SESN3, ZNF671, HBEGF, FITM2, TSHZ2, MEI1, LOC100287290, ZNF540, ZNF614, KIAA1147, LOC100132356, and RMB12B.

9. The kit of claim 1, comprising a set of probes consisting essentially of probes for detecting presence or absence of one or more single polynucleotide polymorphism listed Table 5.

10. A method of diagnosing sarcoidosis in a person, the method comprising measuring expression levels of one or more genes listed in Table 3 and/or the presence or absence of one or more single polynucleotide polymorphism listed in Table 5 in a nucleic acid-containing sample from the person using the kit of claim 1, a relative increase or decrease in expression levels of the one or more genes in Table 3 or the presence or absence of one or more single polynucleotide polymorphism listed in Table 5 indicating the person has or is at risk of developing sarcoidosis.

11. The method of claim 10, wherein the expression level of at least one gene selected from SAP30, CX3CR1, FKBP1A, SERTAD1, APOBEC3D, KLRB1, CR1P1, NOG, SESN3, and ZNF671 is measured, a relatively high level of expression of SAP30 and/or a relatively low level of expression of CX3CR1, FKBP1A, SERTAD1, APOBEC3D, KLRB1, CR1P1, NOG, SESN3 or ZNF671 indicating that the person has or is at risk of developing sarcoidosis.

12. The method of claim 11, wherein the method comprises measuring the expression levels of from two to ten gene selected from SAP30, CX3CR1, FKBP1A, SERTAD1, APOBEC3D, KLRB1, CR1P1, NOG, SESN3, and ZNF671.

13. The method of claim 11, further comprising measuring the expression levels of at least one gene selected from HBEGF, FITM2, TSHZ2, MEI1, LOC100287290, ZNF540, ZNF614, KIAA1147, LOC100132356, and RMB12B, a relatively high level of expression of HBEGF and/or a relatively low level of expression of FITM2, TSHZ2, MEI1, LOC100287290, ZNF540, ZNF614, KIAA1147, LOC100132356, and/or RMB12B, indicating that the person has or is at risk for developing sarcoidosis.

14. The method of claim 13, comprising measuring expression levels of HBEGF, SAP30, FITM2, TSHZ2, MEI1, LOC100287290, ZNF540, ZNF614, KIAA1147, LOC100132356, CX3CR1, RBM12B, FKBP1A, SERTAD1, APOBEC3D, KLRB1, CRIP1, NOG, SENS3, and ZNF671, a relatively high level of expression of HBEGF and SAP30 and a relatively low level of expression of FITM2, TSHZ2, MEI1, LOC100287290, ZNF540, ZNF614, KIAA1147, LOC100132356, CX3CR1, RBM12B, FKBP1A, SERTAD1, APOBEC3D, KLRB1, CRIP1, NOG, SENS3, and ZNF671 indicating that the person has or is at risk for developing sarcoidosis.

15. The method of claim 11, wherein a relatively high level of expression of SAP30 and/or a relatively low level of expression of CX3CR1, FKBP1A, SERTAD1, APOBEC3D, KLRB1, CR1P1, NOG, SESN3 or ZNF671 indicates that the person has or is at risk of developing complicated sarcoidosis.

16. The method of claim 13, wherein a relatively high level of expression of HBEGF and/or a relatively low level of expression of FITM2, TSHZ2, MEI1, LOC100287290, ZNF540, ZNF614, KIAA1147, LOC100132356, and/or RMB12B, indicates that the person has or is at risk for developing complicated sarcoidosis.

17. The method of claim 10, comprising detecting the presence of a single nucleotide polymorphism from the group of single polynucleotide polymorphisms listed in Table 5 in a nucleic acid sample from the person, the presence of one of the single polynucleotide polymorphism listed in Table 5 in the sample indicating that the person is at increased risk for developing sarcoidosis.
